(19) United States
(12) Patent Application Publication
(10) Pub. No.: US 2008/0214887 A1
(43) Pub. Date: Sep. 4, 2008

(54) Brachytherapy apparatus and method using off-center radiation source

(76) Inventors: Joseph A. Heanue, Oakland, CA (US); Paul A. Lovoi, Saratoga, CA (US); Steve Axelrod, Los Altos, CA (US); James E. Jervis, Atherton, CA (US)

Correspondence Address: Thomas M. Freiburger, P.O. Box 1026, Tiburon, CA, 94920, US

(21) Appl. No.: 12/082,492

(22) Filed: Apr. 10, 2008

Related U.S. Application Data
(63) Continuation-in-part of application No. 12/012,010, filed on Jan. 29, 2008.
Application No. 12/012,010 is a continuation-in-part of application No. 10/464,140, filed on Jun. 18, 2003, now Pat. No. 7,322,929.
Application No. 10/464,140 is a continuation-in-part of application No. 11/925,200, filed on Oct. 26, 2007.

(51) Int. Cl. A61M 36/04

(52) U.S. Cl. 600/3

(57) ABSTRACT

A brachytherapy applicator and method of use involve source guides that assume a desired curving, non-linear configuration. A flexible source catheter follows the shape of the source guides when inserted therein. Radiation dose received in various tissue areas can be better controlled using the invention, and the ratio of cavity surface dose to prescription depth dose can be lowered. With sequential manipulation of the source via movement of the catheter, the applicator can deliver radiotherapy to a treatment plan with local variation to prevent overdose, through either stepped or continuous movement of the source. Source guides can be fixed in position and arranged in bowed configuration around a generally central balloon axis, either attached to the balloon wall or not, and the series of off-center guides can be used to shape the dose delivered.

### BACKGROUND OF THE INVENTION

This is a continuation-in-part of application Ser. No. 12/012,010, filed Jan. 29, 2008, which is a continuation-in-part of application Ser. No. 10/464,140, filed Jun. 18, 2003, now U.S. Pat. No. 7,322,929, and application Ser. No. 11/925,200, filed Oct. 26, 2007. The disclosures of both are fully incorporated herein by reference.

This invention concerns radiation therapy, especially brachytherapy, for treating tissues which may have diffuse proliferative disease.

In brachytherapy, a radiation source or a plurality of sources are generally placed within a surgically created or naturally occurring cavity in the body. In particular, this invention relates to delivery of radiation therapy to tissue as might be found in the human breast, or to other tissue, preferably by activation of a miniature, electronic x-ray source. Such therapy often follows surgical treatment of cancer.

Radiation therapy following tumor resection or partial resection is generally administered over a period of time in partial doses, or fractions, the sum of which comprises a total prescribed dose. This fractional application takes advantage of cell recovery differences between normal and cancerous tissue whereby normal tissue tends to recover between fractions, while cancerous tissue tends not to recover.

With conventional brachytherapy, a prescribed dose is selected by the therapist to be administered to a volume of tissue (the target tissue) lying outside the treatment cavity, into which a single radiation source will be placed. Generally the prescribed dose will specify a uniform minimum dose to be delivered at a preferred depth outside the treatment cavity (the prescription depth). Also with conventional brachytherapy, since by the laws of physics radiation intensity falls off, most often exponentially, with increasing distance from the radiation source, it is generally desirable to create and maintain a space between the source of radiation and the first tissue surface to be treated (generally the cavity wall) in order to moderate the absorbed dose at the cavity surface in relation to the prescribed dose delivered at the prescription depth. This is usually accomplished by placing an applicator in the cavity which both fills and shapes the cavity into, most often, a solid figure of revolution (e.g., a sphere or ellipsoid) and positions the radiation source within a source guide situated along a central axis of the cavity so formed and through which the source may be traversed. If the applicator comprises a balloon to shape the cavity, it is preferably inflated using a fluid medium which has radiation attenuation properties similar to those of soft tissue. Water is such a medium. This choice of medium simplifies treatment planning.

Treatment planning is generally automated and is a process whereby system elements are arranged and controlled so as to deliver treatment from a radiation source to target tissue conforming to a dose prescription in an optimal manner. With the apparatus described above, the transverse distance from the source guide on the axis of the cavity to the surface of the cavity varies as the source is traversed through the source guide within the balloon. This creates differences in delivered dose, both from the effects of changing distance as well as from attenuation through varying amounts of inflation medium. These effects do not vary in the same manner as one another, and the combined variation complicates the treatment planning process significantly, particularly when the emissions or isodose patterns of the source are not truly isotropic and their emission characteristics must be accommodated in coordination with the other variations outlined above. Even with automated optimization as part of the planning process, the accuracy of dose delivery may be less than desired.

Furthermore, since the radiation intensity falls off exponentially with increasing distance from the source, when the size of the resection cavity is small, the dose incident on the resection cavity surface may be too great and may risk substantial tissue necrosis if a prescription dose is delivered at the prescription depth. Radiation overdose is to be avoided if at all possible.

One accepted standard in current brachytherapy practice is a prescription depth of one centimeter beyond the treatment cavity surface, thus defining the target tissue, which is used for treatment planning. Assuming the tissue at the prescription depth receives the desired minimum dose, the tissue nearest the source (generally the cavity surface) should not receive more than 2.5 to 3 times the prescription dose (this is the allowable dose ratio). Current standards also require that the skin not receive a dose of more than about 1.5 times the prescription dose. With a one centimeter prescription depth, this usually requires the skin be at least 6-8 mm away from the surface of an applicator engaged against the tissue in the cavity, for a typically sized applicator and cavity. A distance of less than about 6-8 mm may result in doses higher than 1.5 times the prescription dose which are known often to result in undesirable patient cosmesis. Similar complications arise in proximity to bone and other tissues/organs as well. These proximity problems commonly arise and are a contra-indication for conventional isotropic brachytherapy and further complicate the planning process and dose accuracy.

In order to assess distances from cavity surfaces to skin surfaces or to other radiation sensitive structures and to assure cavity shape and contact with the applicator is correct, imaging of the cavity and apparatus is carried out as part of the planning process. Conventional x-ray imaging or CT scanning is often used for this purpose. If, as is often the case, some distances are found to be inadequate, and cannot be overcome, brachytherapy as a treatment modality for the particular patient in question might have to be abandoned.

It is apparent that methods and apparatus are needed that address the complexities described above, simplify the planning process, improve the absorbed dose profile for use with small cavities, and make the therapy more precise, all of which would make brachytherapy an option for a greater proportion of the patient population, and more effective when applied.

In the prior art, Winkler U.S. Pat. No. 6,482,142 describes an applicator to produce an asymmetric radiation pattern in target tissue surrounding a surgical resection cavity. The patent discloses an applicator that holds radioactive isotope “seeds” in an off-axis pattern within the applicator balloon in order to produce asymmetric isodose curves with respect to the balloon volume.

### SUMMARY OF THE INVENTION

The preferred radiation sources for the system of this invention are electronic x-ray sources, the output of which can be either isotropic or directional (side-firing; emitting throughout a solid angle), which can be modulated with regard to radiation penetration (voltage), intensity (current), and/or which can be switched on and off at will. Such x-ray tubes are well known in the art. One reference describing the principles and construction of such tubes is Atoms, Radiation and Radiation Protection, Second Edition, John E. Turner, Ph.D., CHP, 1995, John Wiley & Sons, Section 2.10. Directional source emissions can also be produced by selective shielding of isotropic x-ray sources following the methods described in application Ser. Nos. 11/471,277 and 11/471,013, incorporated herein in their entirety by reference, and in fact, such shielding methods can even be used to limit isotope seed emissions, thus producing similar patterns to the directional emission patterns of x-ray sources as described above. Isotope sources cannot in principle be modulated, however.

In resecting a tumor, the surgeon customarily creates a cavity which approximates a solid figure of rotation without abrupt changes in cavity surfaces, re-entrant features or tissue structures attached to but dangling from the cavity surfaces. An applicator of a predetermined shape, but similar (when inflated, if a balloon type) to the cavity shape is chosen for radiotherapy. When placed in the cavity (and inflated if of the balloon type), it is intended to fill the cavity. A tubular shaft extends from the cavity-filling portion of the applicator proximally to a hub to be positioned outside the body. Preferred applicators of this invention are of the balloon type such that the applicator can be introduced into the body cavity through a minimal incision with the balloon deflated, then when properly positioned, the balloon can be inflated to fill the cavity.

Within the tubular shaft of such an applicator, and extending into the balloon, is a source guide comprising a resilient member, normally straight, but which can be deflected to a bowed shape, at least along the length which will be positioned within the balloon. The bowed shape may form spontaneously when the guide is extended through the straight applicator shaft and released into the balloon volume, or it may be bowed in response to stress exerted within the balloon by other apparatus members. Spontaneous bowing can result from use of superelastic Nitinol, for example, according to the teachings of U.S. Pat. No. 4,665,906. Using these methods, the guide can comprise a Nitinol tube, or can comprise a polymeric tube carrying a longitudinal Nitinol member capable of forming the polymer tube spontaneously when released from its straight configuration. Alternatively, a source guide which bows in response to stress might result if, for example, a tubular polymer element is placed through the applicator shaft accompanied by a parallel string member running along the outside of the polymer tube from outside the body, through a ring, loop or other restraint (through which the string can slide) fastened near the proximal end of the balloon, and extending further and fastening to the polyester tube proximate its distal end. The distal end of the tube preferably engages a socket in the distal end of the balloon in a manner permitting rotation of the tube relative to the balloon. When fully inserted into the applicator, restraining the string while pushing on the proximal end of the polyester tube will bow the tube within the balloon volume. Yet another source guide embodiment can be fashioned having a variable bow or other shape, similar to a steerable catheter (e.g., see Enpath Medical, Inc., Plymouth, Minn.). Many such catheters are available and are often controlled by longitudinal wires positioned in a dispersed manner around the circumference of the catheter and pulled differentially to alter the catheter shape. A source guide can be fashioned similarly and controlled statically or dynamically (during treatment) to position a source, placed within and/or traversed internally, through substantially any arbitrary solid figure of revolution, e.g., such as a cylindrical or hour-glass shape.

As an alternative to manipulation of a source guide during treatment, a series of satellite guides, with or without a central guide, may be utilized to shape the emission pattern of the radiation. This arrangement and other apparatus utilizing the same bowed or shaped members within the balloon will occur to those of skill in the art and will be within the scope of the invention.

Since the shape of the balloon and cavity is substantially predetermined by the resection and balloon choice, the bowed shape of the source guide or guides can be fashioned to follow the cavity wall, preferably but not necessarily at a constant distance, with either style of bowed member. When a source positioned within such a bowed guide is translated axially, coordinated rotation of the guide tube by an external manipulator will sweep the source throughout the cavity at a uniform distance from the cavity wall. Thus the distance to the wall, and the amount of attenuating medium between the source and the cavity wall, will be constant; therefore the radiation incident on the cavity wall will be uniform, as will the dose at the prescription depth, although lower than at the wall. The translation and rotation of the source in the bowed guide tube can approximate a spherical source emitting from everywhere on its surface, so dose does not fall off in an inverse square relationship to distance but falls off a small amount with distance because of the spherical geometry. The source, if isotropic, can be partly shielded such that backward emissions (opposed to the preferred direction) may be substantially eliminated.

Importantly, when a small cavity is to be used for brachytherapy as well as in other circumstances, the radiation emissions can be directed away from the nearest portion of the cavity surface. Since the radiation intensity of an isotropic source decreases exponentially with distance, increasing the distance from the source to the tissue at which the radiation is directed has the effect of reducing the distant cavity-surface incident dose in relation to the prescription dose. In the limit, satellite source guides (or a single guide) can be positioned and fastened at or near the surface of the balloon, maximizing the distance to the opposite balloon surface. Again, and only where the source is isotropic, shielding can be applied to the part of the source guide circumference nearest to or in contact with the cavity surface such that radiation emanating from within the guide would be substantially eliminated on the cavity surfaces nearest or immediately adjacent to the radiation source. Where the source is directed and aimed away from nearby cavity surfaces, however, no shielding is necessary to produce the same effect.

If imaging has revealed radiation-sensitive anatomy unacceptably close to the treatment cavity, the treatment plan can include an over-ride which can interrupt the uniform dose delivery process such that sensitive tissues are spared an overdose and risk of tissue necrosis. Alternatively, radiation sensors placed on or within the body near the at-risk structures can provide monitoring, providing outputs to the system controller signaling the need for a locally reduced dose. Such sensors can be placed using adhesives or needle methods, and power and signal communication can be by conventional wiring or by known wireless methods. Such over-ride might take the form of reduced dwell time of the source when directed toward such structures, or where an x-ray source capable of modulation is used, a reduction in penetration distance or dose intensity can be employed, including shut-off of the source.

The source may be traversed through the cavity in either step-wise or continuous fashion, compensated only for quantity of surface area swept by the solid angle as the source reaches pole of the cavity. The path may be helical or may reciprocate first clockwise, then counterclockwise through 360°, stepping axially after each rotation. Alternatively, the guide may be held at a constant angle while the source translates through the length of the balloon, after which the angular orientation is incremented, and the translation repeated. The speed of source traverse may be used as a dose delivery variable, or the source may be modulated, assuming an x-ray source is being used.

With the methods suggested above, planning is simpler, the ratio of dose incident on the cavity surface to prescription dose at prescription depth can be decreased, and dose accuracy can be improved in many instances. The risk of tissue necrosis is thus minimized, and the proportion of patients for which brachytherapy is indicated is increased.

### DESCRIPTION OF THE DRAWINGS

FIG. 1A is a schematic side view of a portion of an inflated balloon applicator of the invention within a resection cavity of a patient, the applicator comprising a self-deploying source guide positioned in the shaft prior to deployment in the applicator balloon.

FIG. 1B is a view similar to that of FIG. 1A, but with the source guide advanced into the volume of the balloon and self-deployed, and with a radiation source on the tip of a source catheter within the source guide.

FIG. 2A is a schematic side view of a portion of an inflated balloon applicator of the invention, comprising a polymeric source guide advanced into a socket at the distal end of the balloon. An actuating string parallels the source guide, and two radiation sensors are shown, one attached to the patient's skin and another proximate a section of bone, both adjacent to the resection cavity.

FIG. 2B is a view similar to that of FIG. 2A, but with the source guide bowed in response pushing the proximal end of the source guide into the applicator while restraining the proximal end of the string.

FIG. 2C is a section where indicated in FIG. 2B showing a source guide with a source and shielding added which attenuates radiation emissions directed toward the axis of the balloon.

FIG. 2D is a section where indicated in FIG. 2B showing a source guide with a source and shielding added which attenuates emissions directed toward the cavity surface nearest the position of the source.

FIG. 3 is a schematic view in perspective showing two similar manipulators, each capable of transmitting both translational and rotational motion in response to computer control, to the source catheter in the case of the left-most manipulator, and the source guide in the case of the right-most.

FIG. 4 shows a typical decay curve of dose rate or intensity as a function of distance from the source in a uniform, water-like attenuation medium.

FIG. 5A depicts schematically in perspective, a steerable source guide controlled by longitudinal wires.

FIG. 5B depicts schematically in longitudinal cross section, the guide of FIG. 5A with phantom arrows indicating translation and rotation within an applicator balloon.

FIG. 6 depicts in schematic longitudinal cross-section, an applicator with six satellite source guides positioned within the applicator balloon.

FIG. 7A shows an isodose pattern for two arbitrary isodose levels resulting from a configuration of six satellite source guides and a central guide where the radiation from one satellite guide has been eliminated.

FIG. 7B shows an isodose pattern similar to that of FIG. 7A, but where there is no central source guide.

FIG. 8A depicts in schematic longitudinal cross-section, an applicator having six satellite source guides external of, but fastened on the balloon surface, and with a central source guide in addition.

FIG. 8B is a cross-section of the balloon of the applicator of FIG. 8A.

### DESCRIPTION OF PREFERRED EMBODIMENTS

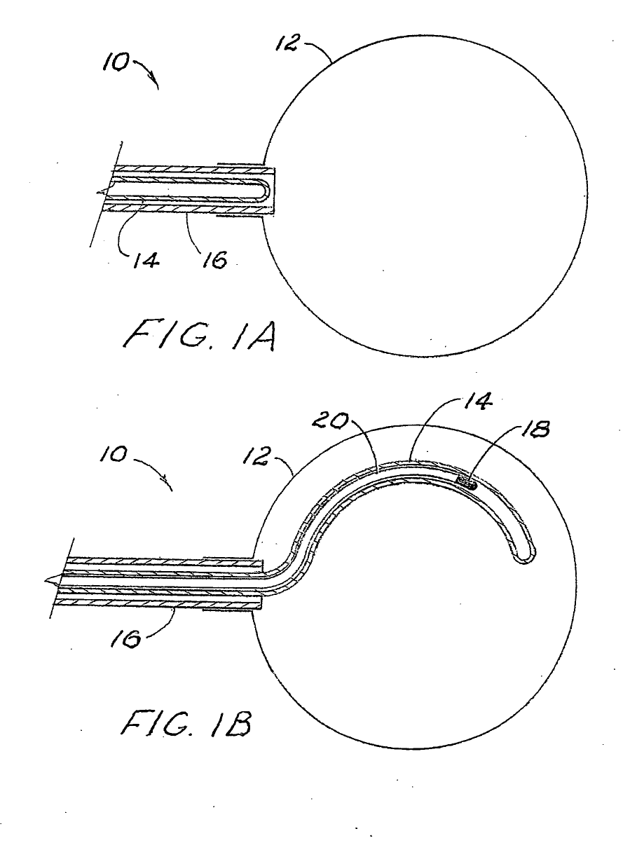

FIG. 1A depicts the balloon portion of an applicator of the invention. The balloon 12 is shown inflated with fluid, preferably by a liquid, filling and shaping the resection cavity C. The tip of a self-deploying source guide 14 is shown positioned within a shaft 16 fixed to the balloon of the applicator, in preparation for advancement into the balloon 12. One material of which such a source guide might be fashioned is superelastic Nitinol. Such a Nitinol guide can be fabricated in a preferred final bowed shape, but when stress is applied, the guide can be forced into another form and restrained in its new shape. When the restraint is removed, the guide will again resume its original shape as fabricated.

In FIG. 1A, the applicator shaft 16 provides the restraint to hold the fabricated shape of the guide 14 in a substantially straight configuration, although the fabricated shape of the guide 14 is a bowed shape along the distal portion which will be inserted into the volume of balloon 10. When the guide is advanced through the shaft into the volume of the balloon, the bow will progressively reform spontaneously, eventually resulting in the shape depicted in FIG. 1B. The distance between the bow and the adjacent cavity surface (within the same longitudinal plane) can be made constant as shown, but need not be.

One alternative to a tubular Nitinol guide is a polymer tube guide with provision for a Nitinol member, for example a wire, which is carried by the polymer tube, but preshaped as described above such that the strength of the Nitinol shapes the polymer tube in the absence of other restraint (for example when the polymer and Nitinol wire are contained within the shaft 16). In such a construction, the Nitinol may be confined to a separate lumen within the polymer guide, or it can also be contained within the source lumen.

In yet another alternative construction, some polymers can be conditioned to behave in a manner similar manner to that of Nitinol as described above by methods familiar to those of skill in the art. An example is polyester. A straight tubular element of polyester can be heat set into a curve with the help of curved fixturing, and allowed to cool. It may then be straightened for insertion into the straight lumen of the shaft 16 for insertion into the cavity of the patient, then subsequently advanced into the volume of balloon 12 where it will resume its curved shape. Methods for such shaping are well known to those of skill in the art.

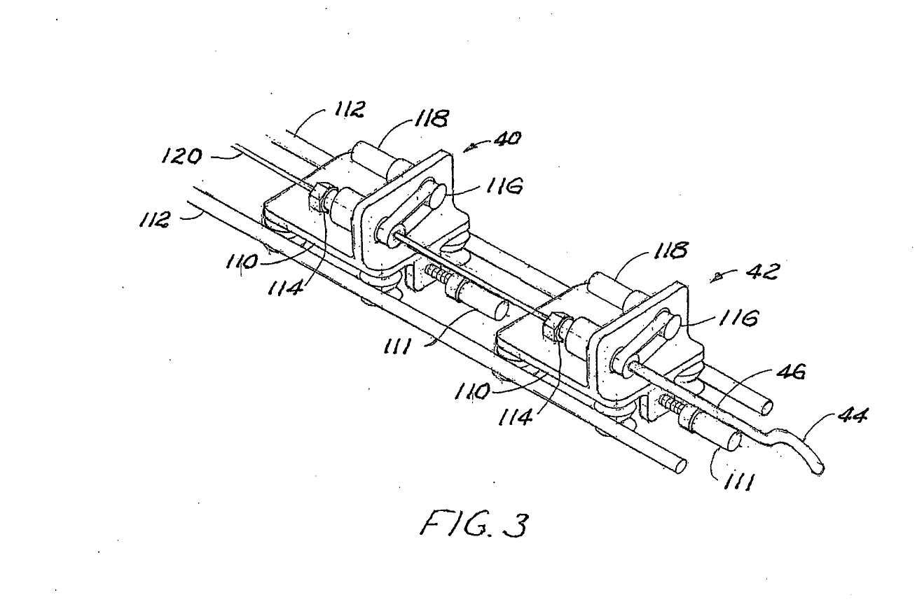

As explained above, FIG. 1B depicts a self-deploying Nitinol source guide 16 advanced into the volume of balloon 12. A source 18 on the end of a source catheter 20 (or optionally a wire) is shown within the source guide 16. Such source catheter 20 on which the source is mounted may be manipulated lengthwise along the axis of the guide 16 under computer control by an axial manipulator responsive to a system controller, all positioned outside the patient (such a manipulator is discussed below and shown in FIG. 3). The source guide 16 may also be rotationally manipulated controllably by a rotational manipulator positioned similarly. By combining translational and rotational motions in a coordinated manner, all portions of the surface of the resection cavity can be exposed to radiation. The details of said coordination will depend on the prescription dose to be delivered, the nature of the source and any shielding, and imposition of any aforementioned over-ride in response to radiation sensitive anatomy proximate to the cavity.

Where the emissions from the radiation source 18 are isotropic and the cavity surface being treated is that nearest the source, the attenuation by the inflation medium opposite the cavity surface being treated (in a sense, behind the emissions of interest) may be inconsequential. If not, the effects of such emissions must be accounted for and included in the treatment planning process. Where the emissions are truly directional, backward emissions can be ignored, but the source catheter 20 and source 18 must be rotated in unison as the source guide is rotated such that the solid angle of emissions continues to address the surface area to be treated, unless the directionality is provided by shielding secured to the guide. One method to assure such directional coordination is to key the catheter rotationally within the source guide, for example by making the lumen of the guide non-circular in cross section, and the outside of the catheter matching in section and size such that, substantially at least, only translation of the catheter within the guide is possible. Alternatively, separate manipulators for catheter and source guide, positioned outside the body and coordinated rotationally by the controller, can achieve the same effect, although differential torsion may require torque resistant construction of catheter and guide in a manner to resist such error. The methods of U.S. Pat. No. 4,425,919 can be employed in this regard. Manipulation of the source may be continuous or intermittent, and rotation can be continuous in one direction, or periodically reversed. Where electronic x-ray sources are employed, periodic reversal of rotation is preferred since that eliminates the need for rotating high-voltage electrical connections. A clockwise 360° rotation followed by counterclockwise reversal followed by a translational step is an example of such preferred manipulation and can be iterated to cover the entire cavity surface. Translation can be simultaneous or sequential, so long as all cavity surfaces are addressed for treatment. Simultaneous movement can be used to generate an essentially helical path of emission. Where the emissions of source 18 are constant, the speed of manipulation can be varied to locally adjust absorbed dose. Where, as with modulated x-ray sources, emissions can be varied, manipulation speed can be constant, or a combination of speed and modulation can be used to accommodate local requirements.

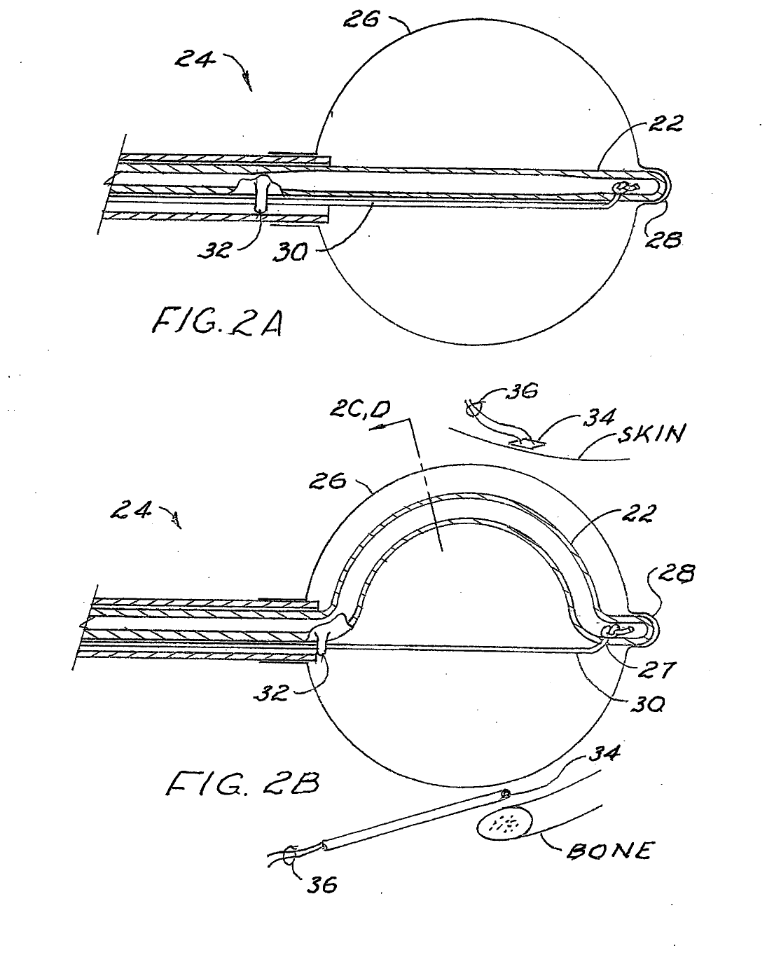

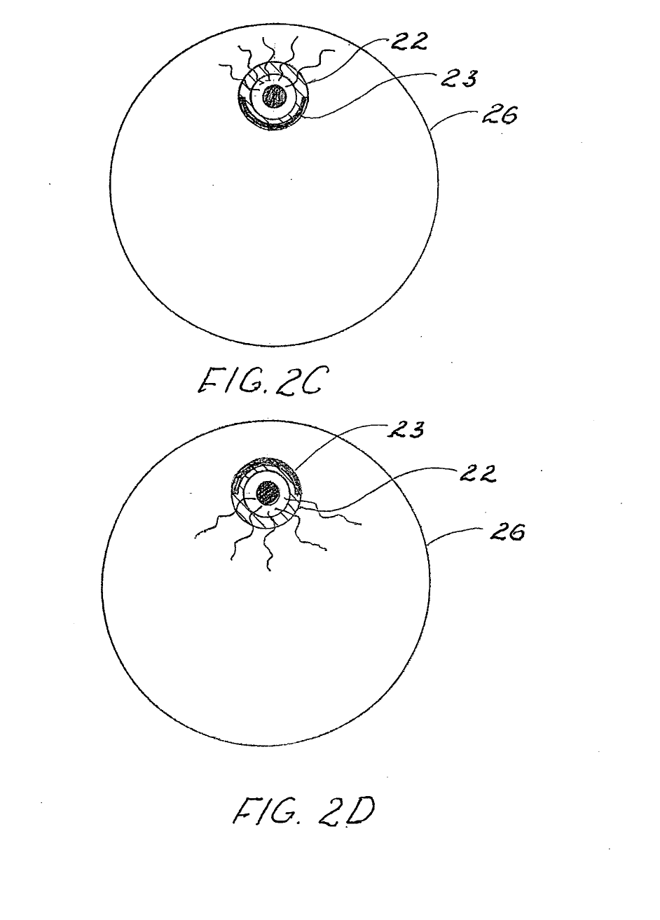

FIG. 2A depicts a different applicator apparatus 24 comprising an alternate embodiment of a source guide 22, and of its support within the balloon 26. The balloon 26 comprises a socket 28 at its distal end to accommodate the distal end of the source guide 22 in a rotating manner. A string 30 is fastened to the guide 22 proximate to its distal tip. The string is led proximally along the outside length of the guide 22, passing through an eye 32 positioned at the point where the proximal end of a bow is to be formed in the guide 22, and onward distally where it is fastened proximate of the distal end of the guide 22. The string is shown passing through a hole 27 into the lumen of the guide 22 where it is knotted. Other fastening methods, for example by bonding, can be used alternatively. The bow portion is to be of resilient construction, as might be provided by use of an engineering polymer, for example of polycarbonate. The distal and proximal straight portions of the guide 22 can be of different materials (e.g., metal, for example stainless steel), or still polycarbonate but of different geometry (e.g., thicker walled) to provide greater rigidity.

In use, the source guide 22 is advanced into the applicator apparatus 24, advancing the string 30 as well, until the distal end of the guide engages the socket 28 at the distal end of the balloon 26. When so engaged, the string 30 is restrained from further advancement from outside the body, but the guide is forced further into the applicator against the resistance of the string. Such advancement forces the bow to form within the balloon volume as shown in FIG. 2B. Advancement is continued until the shape of the bow is as desired. One example of the bow (as shown) is concentric with the shape and at a constant distance from the wall of the balloon 26. Subsequently, a source catheter or wire and a source mounted thereon are introduced into the guide and manipulated in the manner described above in explanation of FIGS. 1A and 1B. Manipulation again may be by apparatus as described above in connection with FIG. 3.

FIGS. 2A and 2B also show radiation sensors 34, for example of the MOSFET type, located on the patient's skin (attached by adhesive for example) and near a segment of bone (positioned by needle for example). Wires 36 are shown which provide communication between the sensors and the system controller. Such sensors, placed near radiation sensitive structures near the resection cavity, can be used to initiate an over-ride on a treatment plan in order to avoid radiation overdose and necrosis of normal tissue. Treatment plan interruption can take the form of an increase in source speed when treating using isotopes, or in the case of electronic x-ray sources, changes in speed, reductions in filament current, or switching off of the x-ray tube, all of which would serve to reduce absorbed dose.

As an alternative to the use of directional sources, substantially similar effects can be obtained practicing the shielding teachings of copending Ser. Nos. 11/471,277 and 11/471,013, incorporated herein by reference in their entirety. By these methods, isotropic x-ray sources and even isotope sources can be made directional, and to some extent modulated by the imposition of elements which are partially attenuating between the source and cavity surface being treated.

As an example, FIG. 2C shows a partial cross section in which the source guide 22 has shielding 23 partially around the circumference of the guide on the side facing the axis of the balloon 26 to attenuate or block radiation emissions on that side of the guide. With this configuration, the radiation is substantially directed toward the cavity surfaces nearest the radiation source.

FIG. 2D is similar, but with the source guide shielding 23 on the side nearest the adjacent cavity surface. With this configuration, the radiation is substantially directed across the diameter of the balloon, through the axis to the far cavity surface. This is useful, particularly where the cavity is small, in that the radiation incident on the far cavity surface is farther removed from the source, hence of lower intensity, while the dose delivered at the prescription depth is held to the prescription. Risk of surface necrosis is thereby reduced, and brachytherapy as a treatment modality is made available where the cavity is small, and where it might otherwise not be practical.

FIG. 3 schematically depicts a manipulator 40 (at left) controlling the source catheter 20a and a similar manipulator 42 (at right) controlling a source guide 14a having bowed section 14b. Both manipulators combine translational and rotational control independently of one another and both are responsive to a central controller (not shown). When combinations of elements or features other than those described in this specific embodiment are used, other translational and rotational manipulators can be devised, some of which may eliminate the need for total or independent control of the catheter 20a and guide 14a, and others of which may be combined into one manipulator.

Each manipulator depicted comprises a sled 110 riding on and confined to rails 112, with its translation actuated by a servo-motor 111. A rotary spindle and collet 114 for gripping the catheter 20a or the guide 14a is mounted on the sled 110 in bearings (not shown), and connected by a belt or gear drive 116 to a servo-motor 118. The catheter 20a (left manipulator) or source guide 14a (right manipulator) thus rotate with their spindles/collets 114. The servos 111 and 118 are responsive to the system controller (not shown) which manages delivery of the treatment plan.

As pictured, the left and right manipulators are capable of being independently controlled, thereby independently positioning the source catheter 20a and source guide 14a, but must be coordinated by the controller to deliver the desired treatment plan. Depending on system requirements, other manipulators may be devised, and such configurations will be apparent to those of skill in the art.

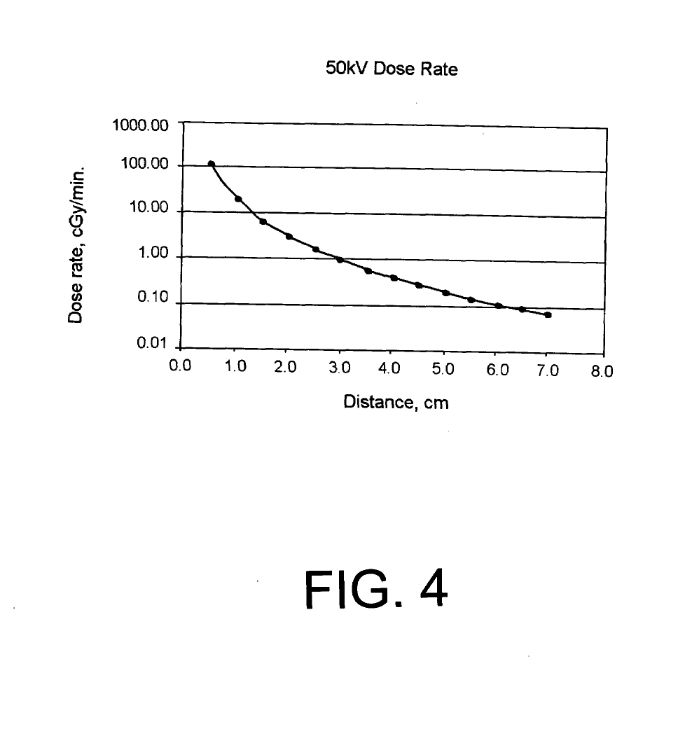

FIG. 4 depicts a typical radiation dose profile for a 50 KV electronic brachytherapy source. The exponential reduction in dose intensity is plotted against distance from the source. Note that the ratio of incident radiation to that one centimeter more distant is lower as one moves to the right on the curve. This illustrates the value of focusing the radiation on tissue across the diameter of the balloon rather than to tissue closer to the source.

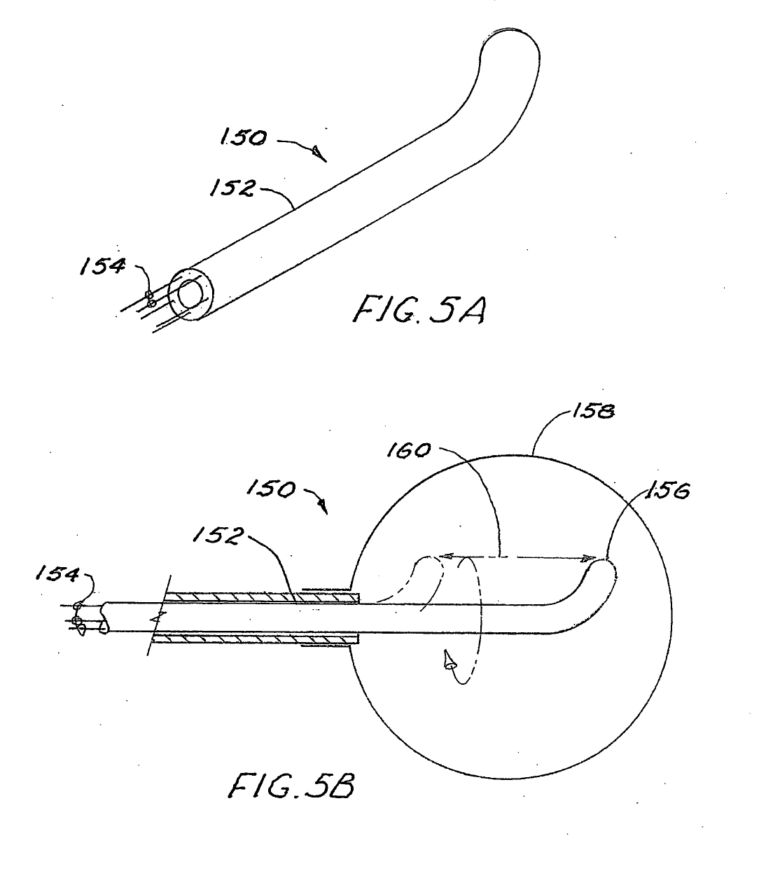

FIG. 5A shows a steerable source guide 150 comprising a tubular, resilient member 152 having longitudinal wires or lines (herein called wires) 154 distributed near the periphery of the guide and slidable in the guide but fixed at the distal end such that when pulled differentially from outside the patient by manipulators responsive to the central controller (manipulator and controller not shown) the guide will assume a desired shape. Such shape may be held statically during translation and/or rotation of the guide 150 within the cavity, or the shape may be changed dynamically during treatment.

FIG. 5B shows the apparatus of FIG. 5A in longitudinal section, with the tip 156 of the guide member 152 positioned within an inflated balloon 158 of an applicator. Such a guide 150 may be translated and rotated within the balloon 158, with variations in wires 154 defining the deflected shape of the guide member 152, which in combination with the translation and rotation of guide 150, will define the shape of the envelope 160 through which the source (not shown) may be swept. The envelope depicted in FIG. 5B is a cylinder as may be seen.

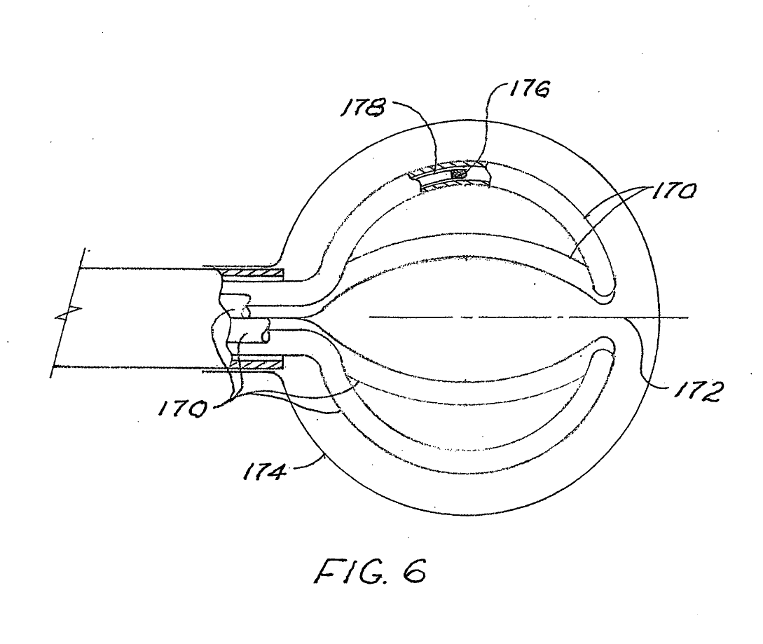

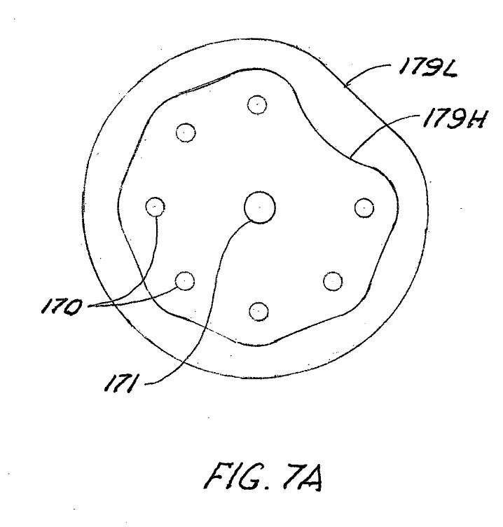

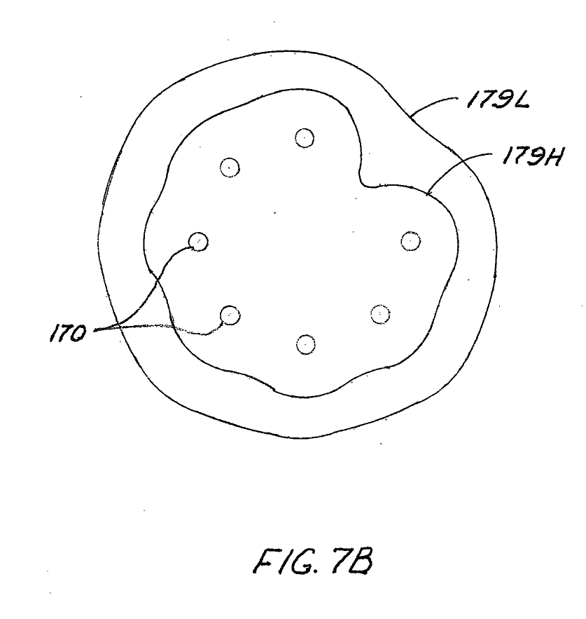

In contrast to manipulation of single source guides as described above, modeling of absorbed dose profiles obtained with a variety of alternate constructions using multiple curved guides positioned around the axis of the applicator balloon has produced several embodiments having important utility. Some of these configurations additionally include a central source guide. FIG. 6 shows in partial longitudinal section, an exemplary schematic arrangement of six guides 170 similar to that shown in FIG. 1B arranged in a uniform pattern around a central axis 172 of the applicator balloon 174. A single radiation source 176 on the end of a catheter 178 is shown in one guide through which it is translated consistent with the treatment plan, after which it would be moved to a different guide in a manner consistent with conventional afterloader practice, and the action repeated sequentially until the therapy or fraction is completed. Alternatively, a series of sources can be manipulated through their respective guides simultaneously producing the same effect.

We have found that when such a satellite configuration as that described in relation to FIG. 6, but with a central guide as well, considerable control of isodose pattern shaping is obtained, particularly if a large proportion of the total absorbed dose comes from the central guide. Where isotropic sources are used, eliminating or modulating one or more sources within their satellite guides 170 serves to reduce total local dose and planning can thus accommodate protection of individual tissue structures near or within the range of the target tissue without significantly disrupting an otherwise uniform prescription. We have found the shadowing effect of such multiple source guide arrangements to be largely negligible because of the relative spacing and sizing of the source and guide elements of the apparatus. The number and positioning of the guides 170 within the balloon 174 can be designed so as to accommodate the treatment plan, and need not be symmetrical about the balloon axis, in contrast to the uniform configuration shown in FIG. 6. Where the satellite guides are unsupported within the volume of the balloon 174, the guides may be fashioned as described above in relation to FIG. 1A. Where the guides can be supported by other apparatus elements, for example the balloon 174, they can be bonded to the balloon or other applicator elements by conventional methods and can follow the balloon wall as the balloon is inflated.

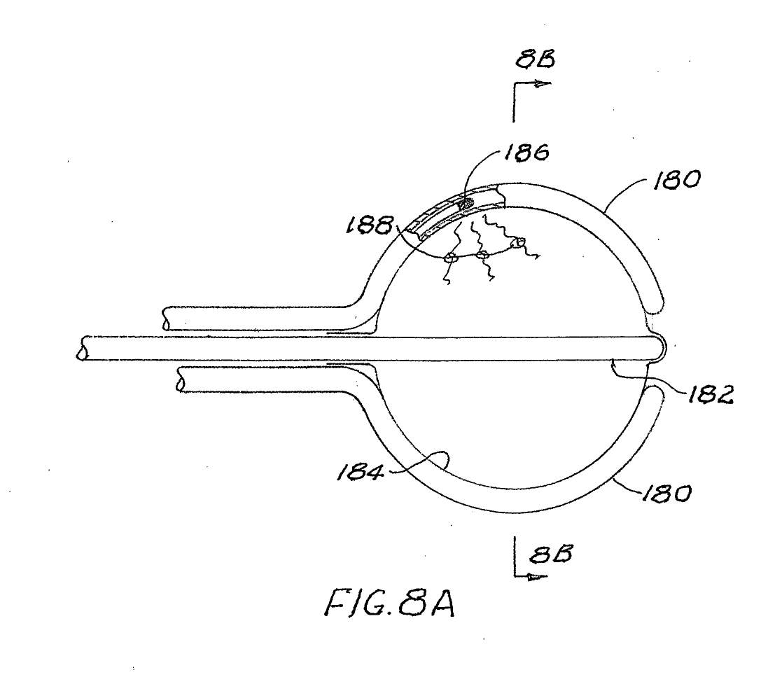

FIG. 7A is an isodose map resulting from computer modeling of an applicator configuration having substantially isotropic sources positioned in satellite guides 170 as shown in FIG. 6, but also with a central isotropic source in a guide 170c emitting about 50% to 60% of the total dose. FIG. 7A shows two arbitrary dose intensity curves for a single pattern of radiation emission from sources in the guides. The inner curve 179H has the higher dose intensity, while the outer 179L has the lower. Radiation from one of eight satellite guide positions is eliminated in FIG. 7A. The total adjacent dose is reduced accordingly.

Note that because the applicator balloon 174 preferably is filled with a liquid having similar attenuation properties as tissue as stated above, the balloon and resection cavity surface do not create a discontinuity in the isodose patterns. In effect, the radiation emitted is attenuated as though passing through a uniform field. Therefore the treatment plan can be fashioned based on distances to deliver the prescribed dose with an acceptable dose ratio to the target tissue as though the balloon and resection cavity surfaces were not present. This illustrates the rationale for using a liquid balloon inflation medium with attenuation properties substantially matched to tissue. If not matched, the problem becomes more complicated, and the position of the balloon/cavity surface becomes important.

FIG. 7B shows an approximation of a similar isodose pattern to that of FIG. 7A, but without the central guide and source. The increased “scallop” effect is readily apparent in the isodose curves 179H and 179L. It is clear that a central source contributing a significant proportion of the total dose contributes importantly to the uniformity of the total delivered dose except where purposeful, local shaping is intended.

Where shielded or directional sources are used, cross-firing (emitting away from the closest tissue and across the balloon volume) can be used to reduce the absorbed dose ratio (surface to prescription depth doses). When used with small balloons and miniature x-ray sources which are easy to shield compared to isotopes, or can be designed to be directional rather than isotropic, this technique is particularly useful, including an embodiment where the source guides are attached to, but are outside the balloon surface. Because of the inherent isotropic nature of isotopic radiation and greater penetration depth of common medical sources, shielding of such sources to create directionality must be more robust and therefore tends to be relatively impractical in such an embodiment.

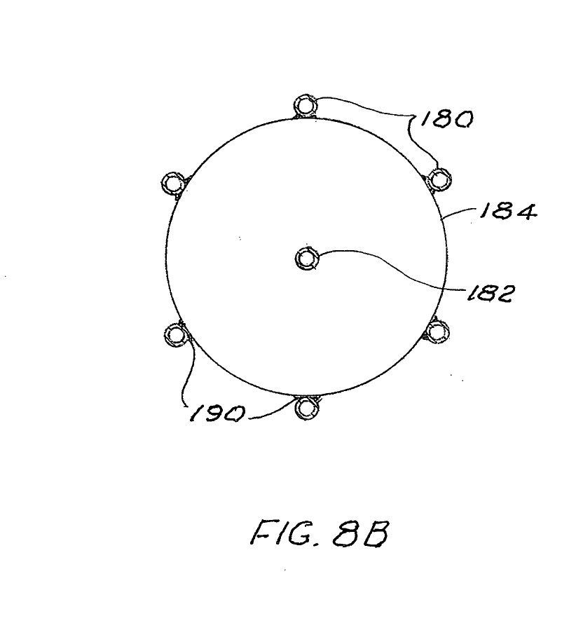

Such an embodiment with source guides positioned outside the balloon 184 is shown in FIG. 8A (a longitudinal cross-section through the balloon) and in FIG. 8B (a transverse cross-section). This embodiment takes maximum advantage of balloon size to create distance between the source or sources and the resection target tissue. In this embodiment, the satellite source guides 180 are resilient, for example of silicone, so as to be easily formed to follow the balloon shape when the balloon is inflated (as in copending application Ser. No. 12/012,010, incorporated herein by reference), and are shown equally spaced around the balloon's outer surface. The spacing can optionally be irregular if desired. Additionally, a central source guide 182 preferably is provided. The sources 186 positioned within the guides 180 are controlled to fire across the balloon 184 to the far tissue surfaces as indicated by the emission lines 188. Again as in the discussion relating to FIG. 6, elimination or modulation of emissions from selected source guides can be used to control local total absorbed dose requirements. In this embodiment, the source guides are bonded conventionally with adhesive 190 (FIG. 8B) to the outer balloon surface.

With directional sources, control of both translation and rotation are necessary to properly direct emissions, in this case across the volume of the balloon. Such manipulation is, for example, enabled by apparatus as described in the discussion of FIG. 3 above. If preferred, this technique can be employed in the absence of a central guide 182, or merely not utilizing a central source as part of the treatment.

Note again that a balloon 184 is shown in FIGS. 8A and 8B at the interior radius of and connecting the satellite guides. Again, however, because of matched attenuation, the emissions behave as though the satellite guides are positioned essentially in a uniformly attenuating field. The dose ratio is determined by the dose intensity at prescription depth across the cavity from the source (which includes the distance diametrally away from the source to the cavity surface plus the further prescription depth), divided by the intensity at the cavity surface. As stated above, this embodiment has particular utility where the cavity is small.

By utilizing the apparatus and methods of this invention, the distance from the source to the cavity surface can be made substantially constant or increased where advantageous. Control of dose distribution and profile is greatly increased. Treatment planning is thereby simplified and delivered dose characteristics are improved. Furthermore, practice of the invention makes brachytherapy an attractive alternative for a greater population of patients than previously possible.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A method for controlling a pattern of radiation dose from a brachytherapy applicator positioned within a patient's body, comprising:
inserting into a cavity of the patient an applicator that has an inflatable balloon, the applicator including a plurality of guides each for receiving a radiation source,
inflating the balloon and deploying the guides such that the guides are in curved configuration and extend outwardly away from a generally central axis of the balloon,
emitting radiation from at least some of the guides using a radiation source positioned therein, so that the radiation delivered to the patient's tissue surrounding the cavity can be substantially in accordance with a prescription dose.

2. The method of claim 1, wherein the plurality of guides in the balloon are substantially symmetrically positioned around the generally central axis of the balloon.

3. The method of claim 2, wherein the plurality of guides are attached to the balloon wall.

4. The method of claim 1, wherein the plurality of guides are attached to the balloon wall.

5. The method of claim 4, wherein the guides are secured to an exterior surface of the balloon wall.

6. The method of claim 4, wherein the guides are secured to an interior surface of the balloon wall.

7. The method of claim 1, wherein a single radiation source is used, and placed and moved through one of said plurality of guides at a time.

8. The method of claim 7, wherein the radiation source is a miniature electronic x-ray source.

9. The method of claim 1, wherein a plurality of radiation sources are used simultaneously in a plurality of the guides.

10. The method of claim 1, wherein fewer than all of the guides are used for emission of radiation via a source.

11. The method of claim 10, wherein the level of radiation emission is substantially the same from all guides used for emission of radiation, and including generating radiation in an isodose pattern which extends generally around the plurality of guides but dips to an inward recess, toward the generally central axis of the balloon, at a side of the balloon where sensitive tissue is located.

12. The method of claim 10, further including emitting radiation from a source within a central guide generally along the generally central axis of the balloon, and wherein radiation emitted from the plurality of guides is directional, directed generally toward the generally central axis of the balloon, so as to generate a radiation isodose pattern that recedes inwardly at a region opposite one or more guides that are not used for emission of radiation.

13. The method of claim 10, wherein one or more selected guides not used are in a position such that sensitive tissues of the patient will receive a lower dose of radiation than adjacent tissue around the cavity.

14. The method of claim 1, wherein the plurality of guides are substantially at a surface of the balloon wall and at generally regular spacing, and the method including using fewer than all of the guides to emit radiation via one or more radiation sources in the guides, so as to create one or more regions around the cavity that receive a lower dose of radiation than adjacent regions around the cavity, thus to avoid damage to sensitive tissue located in such regions receiving lower dose.

15. The method of claim 14, wherein the applicator further includes a central guide aligned generally along the generally central axis of the balloon, and the method including placing a source in the central guide and emitting radiation from the source in the central guide, while also using the peripheral guides to emit radiation so as to create a radiation pattern which is controlled to avoid sensitive tissues adjacent to the cavity.

16. The method of claim 15, wherein the radiation emitted from the source in the central guide is at a different radiation level from the radiation emitted from the plurality of guides.

17. The method of claim 1, wherein radiation from the guides is directional, and the step of emitting radiation from a guide comprising directing the radiation in a direction opposite an area of the balloon wall nearest the guide from which radiation is directed.

18. The method of claim 17, wherein the plurality of guides are substantially at a surface of the balloon wall and at generally regular spacing, and the method including using fewer than all of the guides to emit radiation via one or more radiation sources in the guides, so as to create one or more regions around the cavity that receive a lower dose of radiation than adjacent regions around the cavity, thus to avoid damage to sensitive tissue located in such regions receiving lower dose.

19. The method of claim 17, wherein fewer than all of the guides are used for emission of radiation via a source.

20. The method of claim 19, wherein one or more selected guides not used are in a position such that sensitive tissues of the patient will receive a lower dose of radiation than adjacent tissue around the cavity.

21. A balloon brachytherapy applicator, comprising:
an applicator shaft with an inflatable balloon secured near a distal end of the shaft,
the balloon having a series of guides, each sized to receive a radiation source and each bowing outwardly from a generally central axis of the balloon,
an inflation lumen extending along the shaft to the interior of the balloon for inflation of the balloon after insertion into a patient, and
the shaft carrying a lumen reaching to each guide to facilitate insertion of a radiation source into each of the guides,
whereby the series of bowing guides can be used to control a pattern of radiation delivered to tissue surrounding the applicator from radiation sources in some or all of the guides.

22. The applicator of claim 21, wherein the series of guides are inside the balloon.

23. The applicator of claim 21, wherein the series of guides are each attached to the interior wall of the balloon.

24. The applicator of claim 21, wherein the series of guides are each attached to the exterior wall of the balloon.

25. The applicator of claim 21, wherein the series of guides are positioned in an array about the generally central axis of the balloon and generally equally angularly spaced in the array.

26. The applicator of claim 21, including at least one directional radiation source within at least one of the series of guides, the directional source being positioned to direct radiation toward the generally central axis of the balloon and thus toward a portion of the balloon wall opposite the source.

27. The applicator of claim 21, further including a central guide generally along the generally central axis of the balloon and configured to receive a radiation source.
